(19) United States
(12) Patent Application Publication
(10) Pub. No.: US 2023/0285070 A1
(43) Pub. Date: Sep. 14, 2023

(54) END EFFECTOR ASSEMBLY WITH THIN SEAL PLATES

(71) Applicant: Covidien LP, Mansfield, MA (US)

(72) Inventors: James D. Allen, IV, Broomfield, CO (US); William E. Robinson, Boulder, CO (US); Hector A. Macpherson, Denver, CO (US); Brendan J. Heinig, Boulder, CO (US); Kenneth E. Netzel, Loveland, CO (US)

(21) Appl. No.: 18/016,948

(22) PCT Filed: Jul. 12, 2021
(86) PCT No.: PCT/US21/41266

§ 371 Date: Jan. 19, 2023

Related U.S. Application Data
(60) Provisional application No. 63/056,113, filed on Jul. 24, 2020.

(51) Int. Cl. A61B 18/14

(52) U.S. Cl. CPC A61B 18/1445; A61B 2018/00077

(57) ABSTRACT

A jaw member for a surgical instrument includes a jaw housing and a first electrically conductive material deposited atop the jaw housing. An electrical wire is operably associated with the jaw housing and includes an exposed, electrically conductive tip. The exposed, electrically conductive tip is disposed atop the first electrically conductive material. A second electrically conductive material is deposited atop the exposed, electrically conductive tip to secure the exposed, electrically conductive tip in electrical contact with the first electrically conductive material.

### FIELD

The present disclosure relates to surgical instruments and, more particularly, to electrosurgical instruments for sealing and cutting tissue, and methods of manufacturing same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a jaw member for a surgical instrument that includes a first electrically conductive material deposited atop the insulative jaw housing and an electrical wire operably associated with the insulative jaw housing and including an exposed, electrically conductive tip. The exposed, electrically conductive tip is disposed atop the first electrically conductive material. A second electrically conductive material is deposited atop the exposed, electrically conductive tip to secure the exposed, electrically conductive tip in electrical contact with the first electrically conductive material.

In aspects according to the present disclosure, the first electrically conductive material is selected from the group consisting of aluminum, copper, chromium, titanium, stainless steel, nickel, chrome, tin, platinum, zinc, palladium, gold, nichrome, and a ferritic iron-chromium-aluminum alloy (FeCrAl alloy) sold under the trademark Kanthal®. In other aspects according to the present disclosure, the first and second electrically conductive materials are made from the same material.

In aspects according to the present disclosure, the first or the second electrically conductive material is made from a combination of electrically conductive materials selected from the group consisting of aluminum, copper, chromium, titanium, stainless steel, nickel, chrome, tin, platinum, zinc, palladium, gold, nichrome, and/or Kanthal®.

In other aspects according to the present disclosure, the first electrically conductive material includes a thickness in the range of about 5 microns to about 15 microns.

In aspects according to the present disclosure, the first or second electrically conductive material includes two materials selected from the group consisting of aluminum, copper, chromium, titanium, stainless steel, nickel, chrome, tin, platinum, zinc, palladium, gold, nichrome, and/or Kanthal®, the first material having a thickness in the range of about 5 microns to about 15 microns and the second material having a thickness in the range of about 10 microns to about 130 microns. In other aspects according to the present disclosure, the first or second electrically conductive material includes multiple materials selected from the group consisting of aluminum, copper, chromium, titanium, stainless steel, nickel, chrome, tin, platinum, zinc, palladium, gold, nichrome, and/or Kanthal®, the total thickness of the multiple materials having a thickness in the range of about 10 microns to about 130 microns.

Provided in accordance with aspects of the present disclosure is a jaw member for a surgical instrument that includes an insulative jaw housing and an electrical wire operably associated with the insulative jaw housing and including an exposed, electrically conductive tip. The exposed, electrically conductive tip is disposed atop the jaw housing. An electrically conductive material is deposited atop the insulative jaw housing and is configured to secure the exposed, electrically conductive tip in electrical continuity therewith.

In aspects according to the present disclosure, the electrically conductive material is selected from the group consisting of aluminum, copper, chromium, titanium, stainless steel, nickel, chrome, tin, platinum, zinc, palladium, gold, nichrome, and/or Kanthal®. In other aspects according to the present disclosure, the electrically conductive material is made from a combination of electrically conductive materials selected from the group consisting of aluminum, copper, chromium, titanium, stainless steel, nickel, chrome, tin, platinum, zinc, palladium, gold, nichrome, and/or Kanthal®.

In aspects according to the present disclosure, the electrically conductive material includes a thickness in the range of about 5 microns to about 100 microns. In other aspects according to the present disclosure, the combination of electrically conductive materials includes a thickness in the range of about 10 microns to about 100 microns.

Provided in accordance with aspects of the present disclosure is a jaw member for a surgical instrument that includes an insulative jaw housing having a slot defined therein configured to receive an exposed, electrically conductive tip of an electrical cable. An upper portion of the exposed, electrically conductive tip of the electrical cable is configured to remain exposed when positioned within the slot. An electrically conductive material is deposited atop the insulative jaw housing and is configured to secure the exposed, electrically conductive tip of the cable within the slot and is configured to electrically engage the upper portion of the exposed, electrically conductive tip to provide electrical continuity therebetween.

In aspects according to the present disclosure, the electrically conductive material encloses the exposed, electrically conductive tip within the slot. In other aspects according to the present disclosure, the electrically conductive material is selected from the group consisting of aluminum, copper, chromium, titanium, stainless steel, nickel, chrome, tin, platinum, zinc, palladium, gold, nichrome, and/or Kanthal®.

In aspects according to the present disclosure, the electrically conductive material is made from a combination of electrically conductive materials selected from the group consisting of aluminum, copper, chromium, titanium, stainless steel, nickel, chrome, tin, platinum, zinc, palladium, gold, nichrome, and/or Kanthal®.

In aspects according to the present disclosure, the electrically conductive material includes a thickness in the range of about 5 microns to about 100 microns. In other aspects according to the present disclosure, the combination of electrically conductive materials includes a thickness in the range of about 10 microns to about 100 microns.

Provided in accordance with aspects of the present disclosure is a method of manufacturing a jaw member having a thin tissue-sealing plate and includes: depositing a first electrically conductive material atop a jaw housing; positioning an exposed, electrically conductive tip of an electrical cable atop the first electrically conductive material; and depositing a second electrically conductive material atop the exposed, electrically conductive tip of the electrical cable to secure the exposed, electrically conductive tip in electrical engagement with the first electrically conductive material.

In aspects according to the present disclosure, depositing the first electrically conductive material atop the jaw housing includes sputtering, thermal evaporation, cathodic arc deposition, pulsed laser deposition, electron beam deposition, electroless strike plating, electroplating and/or masking. In other aspects according to the present disclosure, depositing the second electrically conductive material atop the exposed, electrically conductive tip includes sputtering, thermal evaporation, cathodic arc deposition, pulsed laser deposition, electron beam deposition, electroless strike plating, electroplating and/or masking.

In aspects according to the present disclosure, the first or second electrically conductive material is selected from the group consisting of aluminum, copper, chromium, titanium, stainless steel, nickel, chrome, tin, platinum, zinc, palladium, gold, nichrome, and/or Kanthal®. In other aspects according to the present disclosure, the first or second electrically conductive material is made from a combination of electrically conductive materials selected from the group consisting of aluminum, copper, chromium, titanium, stainless steel, nickel, chrome, tin, platinum, zinc, palladium, gold, nichrome, and/or Kanthal®.

In aspects according to the present disclosure, the first electrically conductive material is deposited atop the insulative jaw housing with a thickness in the range of about 5 microns to about 15 microns. In other aspects according to the present disclosure, the second electrically conductive material is deposited atop the exposed, electrically conductive tip with a thickness in the range of about 10 microns to about 130 microns. In yet other aspects according to the present disclosure, the combination of electrically conductive materials is deposited atop the insulative jaw housing with a thickness in the range of about 10 microns to about 130 microns. In still other aspects according to the present disclosure, the total thickness of the first or second electrically conductive materials whether manufactured from one or a combination of materials is less than about 130 microns.

Provided in accordance with additional aspects of the present disclosure is a method of manufacturing a jaw member having a thin tissue-sealing plate and includes: positioning an exposed, electrically conductive tip of an electrical cable atop an insulative jaw housing; and depositing an electrically conductive material atop the exposed, electrically conductive tip of the electrical cable to secure the exposed, electrically conductive tip in electrical engagement with the electrically conductive material.

In aspects according to the present disclosure, depositing the electrically conductive material atop the jaw housing includes sputtering, thermal evaporation, cathodic arc deposition, pulsed laser deposition, electron beam deposition, electroless strike plating, electroplating and/or masking. In other aspects according to the present disclosure, the electrically conductive material is made from a combination of electrically conductive materials selected from the group consisting of aluminum, copper, chromium, titanium, stainless steel, nickel, chrome, tin, platinum, zinc, palladium, gold, nichrome, and Kanthal®.

In aspects according to the present disclosure, the electrically conductive material is deposited atop the insulative jaw housing with a thickness in the range of about 5 microns to about 100 microns. In other aspects according to the present disclosure, the combination of electrically conductive materials is deposited atop the insulative jaw housing with a thickness in the range of about 10 microns to about 130 microns.

Provided in accordance with additional aspects of the present disclosure is a method of manufacturing a jaw member having a thin tissue-sealing plate and includes: inserting an exposed, electrically conductive tip of an electrical cable within a slot defined within an insulative jaw housing such that an upper portion of the exposed, electrically conductive tip remains exposed; and depositing an electrically conductive material atop the insulative jaw housing and the upper portion of the exposed, electrically conductive tip of the electrical cable to secure the exposed, electrically conductive tip in mechanical engagement within the insulative jaw housing and electrical engagement with the electrically conductive material.

In aspects according to the present disclosure, depositing the electrically conductive material atop the jaw housing includes sputtering, thermal evaporation, cathodic arc deposition, pulsed laser deposition, electron beam deposition, electroless strike plating, electroplating and/or masking. In other aspects according to the present disclosure, the electrically conductive material is made from a combination of electrically conductive materials selected from the group consisting of aluminum, copper, chromium, titanium, stainless steel, nickel, chrome, tin, platinum, zinc, palladium, gold, nichrome, and/or Kanthal®.

In aspects according to the present disclosure, the electrically conductive material is deposited atop the insulative jaw housing with a thickness in the range of about 5 microns to about 100 microns. In other aspects according to the present disclosure, the electrically conductive material is deposited atop the insulative jaw housing with a thickness in the range of about 5 microns to about 100 microns. In still other aspects according to the present disclosure, the combination of electrically conductive materials is deposited atop the insulative jaw housing with a thickness in the range of about 10 microns to about 130 microns.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position;

FIG. 5A is a bottom, perspective view of the first jaw member of the end effector assembly of FIG. 4;

FIG. 5B is a top, perspective view of the second jaw member of the end effector assembly of FIG. 4;

FIG. 6A is a side, schematic view of a first jaw member including a first electrically conductive material deposited atop an insulative substrate with a second electrically conductive material deposited thereon securing an exposed tip of an electrical cable;

FIG. 6B is a side, schematic view of another embodiment of a first jaw member including an electrically conductive material deposited atop an insulative substrate and securing an exposed tip of an electrical cable thereon; and

FIG. 6C is a side, schematic view of another embodiment of a first jaw member including an exposed tip of an electrical cable embedded within an insulative substrate and a first electrically conductive material deposited atop the insulative substrate to both secure the exposed tip of the electrical cable and provide electrical continuity therebetween.

### DETAILED DESCRIPTION

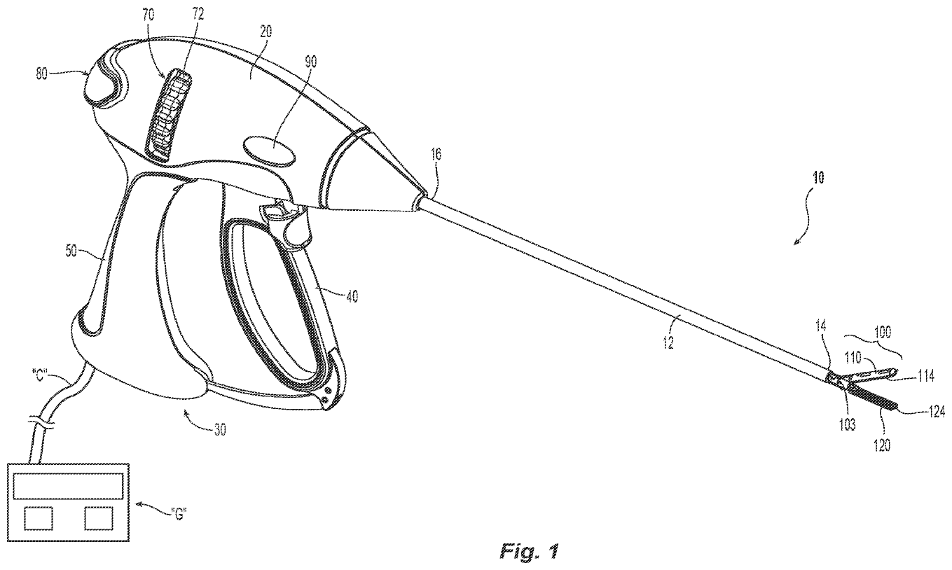

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

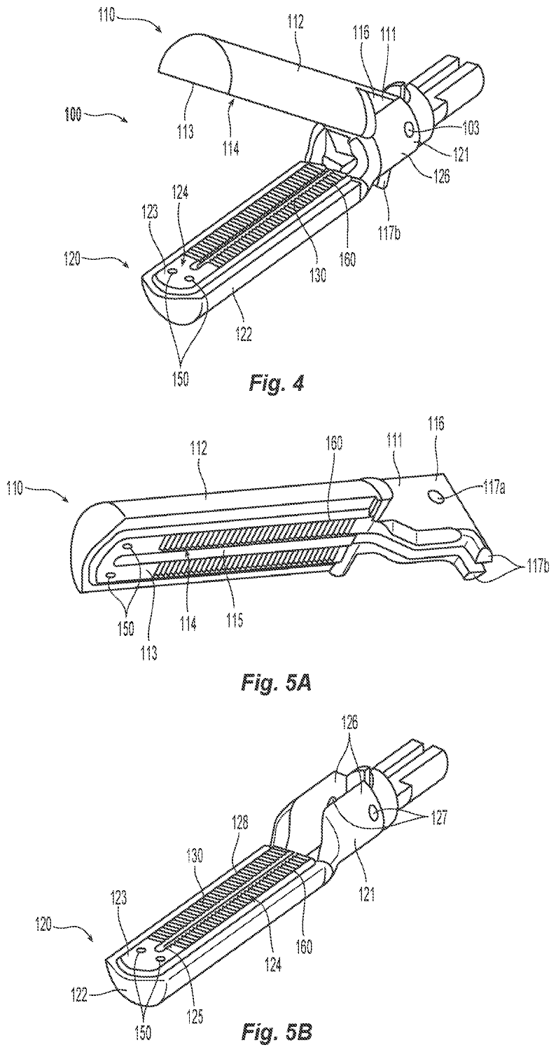

Forceps 10 includes a housing 20, a handle assembly 30, a trigger assembly 60, a rotating assembly 70, a first activation switch 80, a second activation switch 90, and an end effector assembly 100. As shown, end effector assembly 100 includes jaw members 110 and 120 configured for unilateral movement relative to one another. Bilateral movement of the jaw members 110, 120 is also envisioned. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto. First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch 90 is coupled to thermal cutting element 130 of jaw member 120 (FIG. 4) and the electrosurgical generator “G” for enabling the selective activation of the supply of energy to thermal cutting element 150 for thermally cutting tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

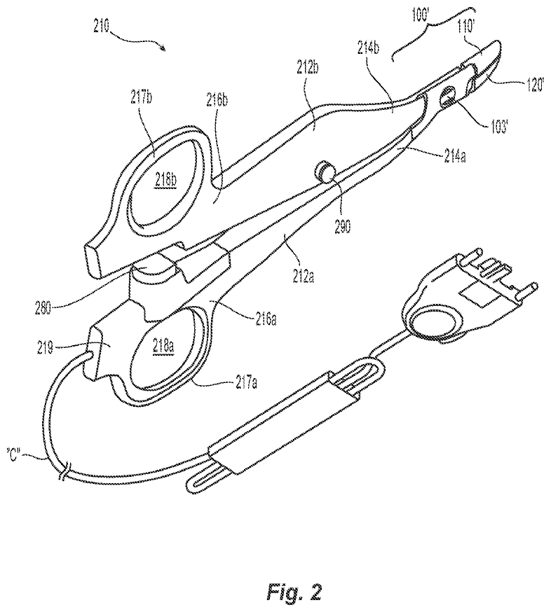

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided for supplying energy to jaw members 110′, 120′ to treat tissue upon sufficient approximation of shaft members 212a, 212b, e.g., upon activation of first activation switch 280 via shaft member 212a. A second activation switch 290 disposed on either or both of shaft members 212a, 212b is coupled to the thermal cutting element (not shown, similar to thermal cutting element 150 of jaw member 120 (FIG. 4)) of one of the jaw members 110′, 120′ of end effector assembly 100′ and to the electrosurgical generator “G” for enabling the selective activation of the supply of energy to the thermal cutting element for thermally cutting tissue.

Jaw members 110′, 120′ define a curved configuration wherein each jaw member is similarly curved laterally relative to a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

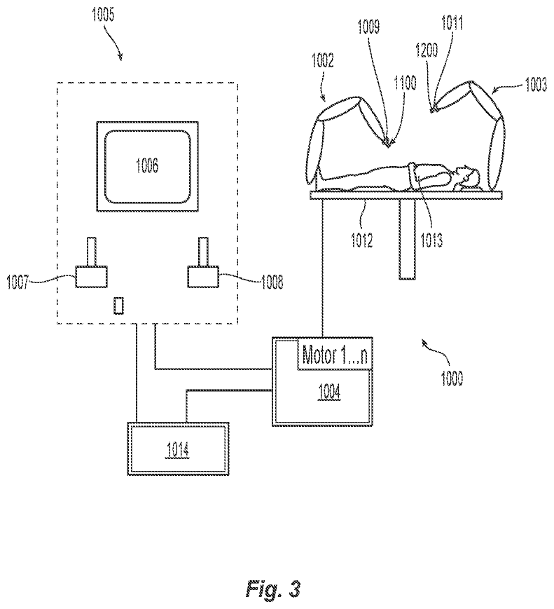

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIG. 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Turning to FIGS. 4-5B, end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include a structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114. An outer surface of the jaw housing 112, in such embodiments, may be at least partially coated with an insulative material or may remain exposed. In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc. Other methods of depositing the tissue-treating plates 113, 123 onto the jaw inserts are described in detail below.

Referring in particular to FIGS. 4 and 5A, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 may further include an aperture 117a for receipt of pivot 103 and at least one protrusion 117b extending therefrom that is configured for receipt within an aperture defined within a drive sleeve of the drive assembly (not shown) such that translation of the drive sleeve, e.g., in response to actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

Regardless of the particular configuration of jaw member 110, jaw member 110 may include a longitudinally-extending insulative member 115 extending along at least a portion of the length of tissue-treating surface 114. Insulative member 115 may be transversely centered on tissue-treating surface 114 or may be offset relative thereto. As explained in more detail below, insulative member 115 may house and electrically and/or thermally isolate a cutter 130 separately activatable to cut tissue upon activation thereof. Further, insulative member 115 may be disposed, e.g., deposited, coated, etc., on tissue-treating surface 114, may be positioned within a channel or recess defined within tissue-treating surface 114, or may define any other suitable configuration. Additionally, insulative member 115 may be substantially (within manufacturing, material, and/or use tolerances) coplanar with tissue-treating surface 114, may protrude from tissue-treating surface 114, may be recessed relative to tissue-treating surface 114, or may include different portions that are coplanar, protruding, and/or recessed relative to tissue-treating surface 114. Insulative member 115 may be formed from, for example, ceramic, parylene, nylon, PTFE, or other suitable material(s) (including combinations of insulative and non-insulative materials).

With reference to FIGS. 4 and 5B, as noted above, jaw member 120 includes a structural frame 121, a jaw housing 122, and a tissue-treating plate 123 defining the tissue-treating surface 124 thereof. Jaw member 120 further includes the thermal cutting element 130 which can be cylindrical in shape or a series of cylinders or other geometrical configurations. Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough to pivotably couple jaw members 110, 120 with one another.

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering), etc. Tissue-treating plate 123, as noted above, defines tissue-treating surface 124. A longitudinally-extending slot or channel 125 is defined through tissue-treating plate 123 and is positioned to oppose insulative member 115 of jaw member 110 (FIG. 5A) in the approximated position. The slot or channel 125 may be defined within an integrally-formed tissue-treating plate 123 or may be defined between two tissue-treating plates that, together, operate as a single treatment surface (not shown) Slot 125 may extend through at least a portion of jaw housing 122, a jaw insert (if so provided), and/or other components of jaw member 120 to enable receipt of thermal cutting element 130 at least partially within slot 125.

Thermal cutting element 130, more specifically, is disposed within longitudinally-extending slot 125 such that thermal cutting element 130 opposes insulative member 115 of jaw member 110 (FIG. 5A) in the approximated position. Thermal cutting element 130 may be configured to contact insulative member 115 (FIG. 5A) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutting element 130 may be surrounded by an insulative member 128 disposed within slot 125 to electrically and/or thermally isolate thermal cutting element from tissue-treating plate 123. Alternatively or additionally, thermal cutting element 130 may include an insulative coating on at least the sides thereof for similar purposes. Thermal cutting element 130 and insulative member 128 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

In embodiments where end effector assembly 100, or a portion thereof, is curved, longitudinally-extending slot 125 and thermal cutting element 130 may similarly be curved, e.g., wherein longitudinally-extending slot 125 and thermal cutting element 130 (or corresponding portions thereof) are relatively configured with reference to an arc (or arcs) of curvature rather than a longitudinal axis. If end effector 100 is curved, longitudinally-extending slot 125 and thermal cutting element 130 may also remain straight or vice versa. Thus, the terms longitudinal, transverse, and the like as utilized herein are not limited to linear configurations, e.g., along linear axes, but apply equally to curved configurations, e.g., along arcs of curvature. In such curved configurations, insulating member 115 of jaw member 110 (FIG. 5A) is likewise curved.

Generally referring to FIGS. 1-5B, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutting element 130.

Thermal cutting element 130, on the other hand, is configured to connect to electrosurgical generator “G” (FIG. 1) and second activation switch 90 to enable selective activation of the supply of energy to thermal cutting element 130 for heating thermal cutting element 130 to thermally cut tissue disposed between jaw members 110, 120, e.g., to cut the sealed tissue into first and second sealed tissue portions. Other configurations including multi-mode switches, other separate switches, etc. may alternatively be provided.

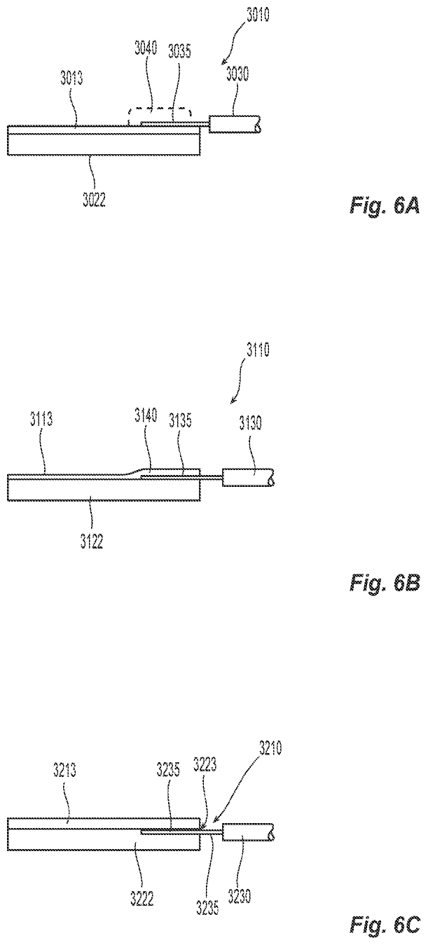

Referring to FIGS. 6A-6C, electrically connecting the thin tissue-sealing plates 113, 123 to an electrical cable (e.g., cable 3030) may be accomplished utilizing a variety of manufacturing methods. For example, FIG. 6A shows one embodiment of a jaw member 3010 electrically connected to an electrical cable 3030. More particularly, jaw member 3010 includes a substrate 3022 having a thin tissue-sealing plate 3013 made from one or more of the above-identified conductive materials disposed or deposited thereon (as described above) for electrically treating tissue. Electrical cable 3030 includes an exposed conductive tip 3035 that is configured to sit atop the thin tissue-sealing plate 3013 such that there is electrical continuity therebetween. A second electrically conductive material 3040 is deposited atop the exposed conductive tip 3035 to mechanically secure the conductive tip 3035 to the underlying conductor 3013, e.g., tissue-sealing plate 3013. In embodiments, the second electrically conductive material 3040 may be positioned proximate the actual surface that contacts tissue of the tissue-sealing plates 3013 since the electrical effects of the second conductor 3040 may affect the quality of the tissue seal. In other embodiments, the second electrically conductive material 3040 has little or no effect on the tissue seal and may be positioned closer to the actual surface that contacts the tissue during a seal.

One or more of the above-identified conductive materials may be disposed atop the exposed conductive tip 3035 to mechanically secure the tip 3035 to tissue-sealing plate 3013. Further, it is contemplated that any of the above-mentioned manufacturing or deposition techniques or processes may be utilized to accomplish this purpose.

FIG. 6B shows another embodiment of a jaw member 3110 electrically connected to an electrical cable 3130. More particularly, jaw member 3110 includes a substrate 3122 having a thin tissue-sealing plate 3113 made from one or more of the above-identified conductive materials disposed or deposited thereon (as described above) for electrically treating tissue. Electrical cable 3130 includes an exposed conductive tip 3135 that is configured to sit atop the underlying substrate 3122 prior to the deposition process (or other, above-described process for affixing a conductive material atop a substrate). The conductive material making up the thin tissue-sealing plate 3113 is deposited atop the exposed conductive tip 3135 to mechanically secure the conductive tip 3135 to the underlying conductor, e.g., tissue-sealing plate 3113. In embodiments, the electrical conductive tip 3135 may be proximally disposed on the tissue-sealing plate 3113.

FIG. 6C shows yet another embodiment of a jaw member 3210 electrically connected to an electrical cable 3230. More particularly, jaw member 3210 includes a substrate 3222 having a thin tissue-sealing plate 3213 made from one or more of the above-identified conductive materials disposed or deposited thereon (as described above) for electrically treating tissue. Electrical cable 3230 includes an exposed conductive tip 3235 that is configured to embed within the substrate 3222 in mechanical connection therewith. The exposed conductive tip 3235 may be secured within a hole 3223 defined within the proximal end of substrate 3222 such that an upper surface 3235′ thereof remains exposed. A slot (not shown) is also contemplated for this purpose. Once the exposed tip 3235 is positioned within the hole (or slot) 3223, the conductive material making up the tissue-sealing plate 3213 is deposited atop the substrate using any of the above-identified techniques. Once, deposited, the exposed tip 3235 electrically couples to the tissue-sealing plate 3213. The hole 3223 may be configured to mechanically secure the conductive tip 3235 in place within the substrate 3222 or the conductive material, once deposited, may be configured to secure the conductive tip 3235 in place within the substrate 3222. In cases where the conductive tip 3235 is disposed within a slot defined in the substrate 3222, the conductive material making up the tissue-sealing surface 3213 partially fills and surrounds the conductive tip 3235 to secure the conductive tip 3235 within the substrate 3222. The hole or slot 3223 may be formed during a prior manufacturing step, e.g., injection molding, overmolding, laser cutting, drilling, etc.

In embodiments, the exposed conductive tip, e.g., tip 3035, may be ultrasonically welded to the tissue-sealing plate 3013 after the tissue-sealing plate 3013 has been deposited or otherwise formed atop the housing 3022. For example, after the thin tissue-sealing plate 3013 is sputtered onto the jaw housing 3022, the exposed conductive tip 3035 may be ultrasonically welded atop the thin tissue-sealing plate 3013. Other mechanical connections are also envisioned, e.g., plug-on or plug-in connections may be formed during the process of depositing or otherwise forming the thin tissue sealing plate 3013.

Turning back to the tissue-sealing plates, for example thin sealing plate 3013 and the various methods of manufacturing the same, it is contemplated that the tissue-sealing plate 3013 may be manufactured in thin layers that are deposited (or otherwise) atop jaw housing 3022 (or jaw inserts). For example, it is contemplated that tissue-sealing plate 3013 may be deposited onto the jaw housing 3022 via one or more of the following vapor deposition manufacturing techniques: sputtering, thermal evaporation, cathodic arcing, pulsed laser deposition, electron beam deposition. Other techniques may include: electroless strike or plating and electro-plating, shadow masking.

Utilizing one or more of these techniques provides a thin layer of conductive material which has the benefit of dissipating heat quickly compared to traditional tissue-sealing plates. Other advantages of thin-layered tissue sealing plates include: ability to heat up quickly, requiring less energy to heat up and maintain heat during the sealing process, ability to seal tissue in a reduced timeframe compared to traditional tissue-sealing plates, and more versatility with tissue seal plate geometry.

It is contemplated that any one of the following materials or combinations thereof may be utilized to form a thin tissue-sealing plate 3013: aluminum, copper, chromium, titanium, stainless steel, nickel, chrome, tin, platinum, palladium, gold, nichrome, and Kanthal®. It is contemplated that during manufacturing, combinations of materials may be utilized for a particular purpose or to achieve a particular result. For example, one material may be utilized as a base conductor with a second material used as an outer or inner conductor to act as the heating element. Additional techniques or materials may be added to act as thermal cutting elements such as those described with reference to U.S. patent application Ser. No. 16/785,347 filed Feb. 7, 2020, U.S. Provisional Patent Application Ser. No. 62/952,232 filed Dec. 21, 2019, U.S. patent application Ser. No. 16/838,551 filed Apr. 2, 2020, and U.S. patent application Ser. No. 16/518,016 filed Jul. 22, 2019, the entire contents of each of which being incorporated by reference herein.

In other embodiments, materials may be mixed during the application process. In some embodiments, the material used (e.g., Aluminum, copper etc.,) may be thin and still promote a good sealing effect while other materials may have to be thicker to produce the same or similar sealing effect due to the particular material's level of continuity and/or conductivity. In this latter instance, a highly conductive base material may be utilized with the thinner less conductive material to produce a desired effect.

In embodiments, a biocompatible material may be utilized to cover a non-biocompatible material. In other embodiments, the materials may be deposited (or otherwise disposed on the jaw member(s)) in non-uniform layers while still allowing for transitions, e.g., side-to-side transitions. The materials could be deposited (or otherwise disposed on the jaw member(s)) in an alternating fashion and more than one electrical circuit may be employed.

Examples of tissue-sealing plates that may be defined as thin tissue-sealing plates may include single layer conductors in the range of about 1 microns to about 100 microns with a nominal thickness of about 30 microns. For example, in one embodiment, a thin layer of sputtered aluminum is deposited onto a jaw housing 3022 to form a thin tissue-sealing plate 3013 of about 30 microns thick. For two or more conductors, thin tissue-sealing plates, the first conductor may be in the range of about 1 microns to about 129 microns and the second conductor may be in the range of about 1 microns to about 130 microns. For multiple conductors, the total thickness may be in the range of about 2 microns to about 130 microns. Non-conductive, electrically transparent, thermally transparent, or electrically and/or thermally porous materials may also be layered in a similar fashion atop, below or between the conductive materials. These materials may be layered atop the jaw housing 3022 within the above ranges.

As mentioned above and with reference to FIGS. 4-5B, the jaw housing 112, 122 (or jaw insert) may act as the support structure for the thin tissue-sealing plates 113, 123. By utilizing any of the above manufacturing techniques, certain aspects of the jaw housing 112, 122 may be masked such that these features act as secondary jaw features, e.g., stop members or textured surfaces for gripping. For example, using microfabrication, a shadow mask or an applied mask may be disposed over one of more aspects of one or both jaw housings 112, 122 during the manufacturing process to form the stop members 150 (FIGS. 4-5B). In another example, one or both jaw housings 112, 122 (or jaw inserts) may be masked in certain areas during application of the conductive material to form the tissue-sealing plates 113, 123 in a similar fashion to act as the grip texture 160 on tissue-treating surfaces 114, 124 (FIGS. 4-5B). The grip texture 160 may be configured to grip tissue in one direction and let tissue slide in another direction to facilitate tissue ejection from the jaw members 110, 120. It is contemplated that during deposition (or other manufacturing techniques) certain areas of the jaw housings 112, 122 may be masked on surfaces parallel to the tissue-sealing plates and/or on surfaces that are non-parallel to the tissue sealing plates 113, 123.

In embodiments, the final tissue-sealing plate 113, 123 may need to be coated with a material to reduce adhesion to tissue, provide different electrical patterns and/or provide different thermal effects. Examples of such materials include oil infused materials, silicone-based organic polymers such as PDMS (HMDSO) materials, parylene, etc.

The present disclosure also relates to a method of manufacturing a jaw member 3010 including a thin tissue-sealing plate 3013. One method includes: depositing a first electrically conductive material 3013 atop a jaw housing 3022; positioning an exposed, electrically conductive tip 3035 of an electrical cable 3030 atop the first electrically conductive material 3013; and depositing a second electrically conductive material 3040 atop the exposed, electrically conductive tip 3035 of the electrical cable 3030 to secure the exposed, electrically conductive tip 3035 in electrical engagement with the first electrically conductive material 3013.

In embodiments, depositing the first electrically conductive material 3013 atop the jaw housing 3022 may include techniques such as sputtering, thermal evaporation, cathodic arc deposition, pulsed laser deposition, electron beam deposition, electroless strike plating, electroplating and/or masking. In another embodiment, depositing the second electrically conductive material 3040 atop the exposed, electrically conductive tip 3035 includes sputtering, thermal evaporation, cathodic arc deposition, pulsed laser deposition, electron beam deposition, electroless strike plating, and/or electroplating. Various masking techniques are also envisioned for applying or depositing the first or additional materials atop the jaw housing 3022.

In embodiments, the first or second electrically conductive material 3013, 3040 is selected from the group consisting of aluminum, copper, chromium, titanium, stainless steel, nickel, chrome, tin, platinum, zinc, palladium, gold, nichrome, and Kanthal®. In other embodiments, the first or second electrically conductive material 3013, 3040 is made from a combination of electrically conductive materials selected from the group consisting of aluminum, copper, chromium, titanium, stainless steel, nickel, chrome, tin, platinum, zinc, palladium, gold, nichrome, and Kanthal®.

In embodiments, the first electrically conductive material 3013 is deposited atop the insulative jaw housing 3022 with a thickness in the range of about 1 microns to about 129 microns thick. In other embodiments, the second electrically conductive material 3040 is deposited atop the exposed, electrically conductive tip with a thickness in the range of about 2 microns to about 129 microns thick.

In embodiments, the combination of electrically conductive materials is deposited atop the insulative jaw housing 3022 with a thickness in the range of about 2 microns to about 130 microns thick. The total thickness of the first or second electrically conductive materials 3013, 3040 whether manufactured from one or a combination of materials should be less than about 130 microns.

The present disclosure also relates to another method of manufacturing a jaw member 3110 having a thin tissue-sealing plate 3113 and includes: positioning an exposed, electrically conductive tip 3135 of an electrical cable 3130 atop an insulative jaw housing 3122; and depositing an electrically conductive material 3113 atop the exposed, electrically conductive tip 3135 of the electrical cable 3130 to secure the exposed, electrically conductive tip 3135 in electrical engagement with the electrically conductive material 3113.

In embodiments, depositing the electrically conductive material 3113 atop the jaw housing 3122 includes sputtering, thermal evaporation, cathodic arc deposition, pulsed laser deposition, electron beam deposition, electroless strike plating, electroplating and/or masking. In other embodiments, the electrically conductive material 3113 is made from a combination of electrically conductive materials selected from the group consisting of aluminum, copper, chromium, titanium, stainless steel, nickel, chrome, tin, platinum, zinc, palladium, gold, nichrome, and Kanthal®.

In embodiments, the electrically conductive material 3113 is deposited atop the insulative jaw housing 3122 with a thickness in the range of about 5 microns to about 100 microns. In other embodiments, the combination of electrically conductive materials is deposited atop the insulative jaw housing 3122 with a thickness in the range of about 10 microns to about 130 microns.

The present disclosure also relates to another method of manufacturing a jaw member 3210 having a thin tissue-sealing plate 3213 and includes: inserting an exposed, electrically conductive tip 3235 of an electrical cable 3230 within a slot 3223 defined within an insulative jaw housing 3222 such that an upper portion 3235′ of the exposed, electrically conductive tip 3235 remains exposed; and depositing an electrically conductive material 3213 atop the insulative jaw housing 3222 and the upper portion 3235′ of the exposed, electrically conductive tip 3235 of the electrical cable 3230 to secure the exposed, electrically conductive tip 3235 in mechanical engagement within the insulative jaw housing 3222 and electrical engagement with the electrically conductive material 3213.

In embodiments, depositing the electrically conductive material 3213 atop the jaw housing 3222 includes sputtering, thermal evaporation, cathodic arc deposition, pulsed laser deposition, electron beam deposition, electroless strike plating, electroplating and/or masking. In other embodiments, the electrically conductive material 3213 is made from a combination of electrically conductive materials selected from the group consisting of aluminum, copper, chromium, titanium, stainless steel, nickel, chrome, tin, platinum, zinc, palladium, gold, nichrome, and Kanthal®.

In embodiments, the electrically conductive material 3213 is deposited atop the insulative jaw housing 3222 with a thickness in the range of about 5 microns to about 100 microns. In other embodiments, the electrically conductive material 3213 is deposited atop the insulative jaw housing 3222 with a thickness in the range of about 5 microns to about 100 microns. In still other embodiments, the combination of electrically conductive materials is deposited atop the insulative jaw housing 3222 with a thickness in the range of about 10 microns to about 130 microns.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for a surgical instrument, comprising:
a jaw housing;
a first electrically conductive material deposited atop the jaw housing;
an electrical wire operably associated with the jaw housing and including an exposed, electrically conductive tip, the exposed, electrically conductive tip disposed atop the first electrically conductive material; and
a second electrically conductive material deposited atop the exposed, electrically conductive tip to secure the exposed, electrically conductive tip in electrical contact with the first electrically conductive material.

2. The jaw member according to claim 1, wherein the first electrically conductive material is selected from the group consisting of aluminum, copper, chromium, titanium, stainless steel, nickel, chrome, tin, platinum, zinc, palladium, gold, nichrome, and ferritic iron-chromium-aluminum alloys.

3. The jaw member according to claim 1, wherein the first and second electrically conductive materials are made from the same material.

4. The jaw member according to claim 1, wherein the first or the second electrically conductive material is made from a combination of electrically conductive materials selected from the group consisting of aluminum, copper, chromium, titanium, stainless steel, nickel, chrome, tin, platinum, zinc, palladium, gold, nichrome, and ferritic iron-chromium-aluminum alloys.

5. The jaw member according to claim 1, wherein the first electrically conductive material includes a thickness in the range of about 1 micron to about 130 microns.

6. The jaw member according to claim 1, wherein the first or second electrically conductive material includes two materials selected from the group consisting of aluminum, copper, chromium, titanium, stainless steel, nickel, chrome, tin, platinum, zinc, palladium, gold, nichrome, and ferritic iron-chromium-aluminum alloys, the first material having a thickness in the range of about 1 micron to about 129 microns and the second material having a thickness in the range of about 1 micron to about 129 microns.

7. The jaw member according to claim 1, wherein the first or second electrically conductive material includes multiple materials selected from the group consisting of aluminum, copper, chromium, titanium, stainless steel, nickel, chrome, tin, platinum, zinc, palladium, gold, nichrome, and ferritic iron-chromium-aluminum alloys, the total thickness of the multiple materials having a thickness in the range of about 2 microns to about 130 microns.

8. A jaw member for a surgical instrument, comprising:
a jaw housing;
an electrical wire operably associated with the jaw housing and including an exposed, electrically conductive tip, the exposed, electrically conductive tip disposed atop the jaw housing; and
an electrically conductive material deposited atop the jaw housing and configured to secure the exposed, electrically conductive tip in electrical continuity therewith.

9. The jaw member according to claim 8, wherein the electrically conductive material is selected from the group consisting of aluminum, copper, chromium, titanium, stainless steel, nickel, chrome, tin, platinum, zinc, palladium, gold, nichrome, and ferritic iron-chromium-aluminum alloys.

10. The jaw member according to claim 8, wherein the electrically conductive material is made from a combination of electrically conductive materials selected from the group consisting of aluminum, copper, chromium, titanium, stainless steel, nickel, chrome, tin, platinum, zinc, palladium, gold, nichrome, and ferritic iron-chromium-aluminum alloys.

11. The jaw member according to claim 8, wherein the electrically conductive material includes a thickness in the range of about 1 micron to about 130 microns.

12. The jaw member according to claim 10, wherein the combination of electrically conductive materials includes a thickness in the range of about 2 microns to about 130 microns.

13. A jaw member for a surgical instrument, comprising:
a jaw housing including a slot defined therein configured to receive an exposed, electrically conductive tip of an electrical cable, an upper portion of the exposed, electrically conductive tip of the electrical cable remaining exposed when positioned within the slot; and
an electrically conductive material deposited atop the jaw housing and configured to secure the exposed, electrically conductive tip of the cable within the slot and configured to electrically engage the upper portion of the exposed, electrically conductive tip to provide electrical continuity therebetween.

14. The jaw member according to claim 13 wherein the electrically conductive material encloses the exposed, electrically conductive tip within the slot.

15. The jaw member according to claim 13, wherein the electrically conductive material is selected from the group consisting of aluminum, copper, chromium, titanium, stainless steel, nickel, chrome, tin, platinum, zinc, palladium, gold, nichrome, and ferritic iron-chromium-aluminum alloys.

16. The jaw member according to claim 13, wherein the electrically conductive material is made from a combination of electrically conductive materials selected from the group consisting of aluminum, copper, chromium, titanium, stainless steel, nickel, chrome, tin, platinum, zinc, palladium, gold, nichrome, and ferritic iron-chromium-aluminum alloys.

17. The jaw member according to claim 13, wherein the electrically conductive material includes a thickness in the range of about 1 micron to about 130 microns.

18. The jaw member according to claim 16, wherein the combination of electrically conductive materials includes a thickness in the range of about 2 microns to about 130 microns.
